(19) United States
(12) Patent Application Publication
(10) Pub. No.: US 2018/0247700 A1
(43) Pub. Date: Aug. 30, 2018

(54) SYSTEM AND METHOD FOR MANAGING THE MEDICAL RECORD OF A PATIENT BY AUTOMATICALLY COLLECTING CLINICAL DATA

(71) Applicant: Inpeco Holding Ltd., Qormi (MT)

(72) Inventor: Gianandrea PEDRAZZINI, Paradiso (CH)

(73) Assignee: Inpeco Holding Ltd., Qormi (MT)

(21) Appl. No.: 15/757,666

(22) PCT Filed: Sep. 7, 2016
(86) PCT No.: PCT/IB2016/055325

§ 371 Date: Mar. 6, 2018

(30) Foreign Application Priority Data
Sep. 7, 2015 (IT) 102015000049081

(51) Int. Cl. G16H 10/60; G16H 20/10

(52) U.S. Cl. CPC G16H 10/60; G16H 20/10

(57) ABSTRACT

A system for managing the medical record of a patient by automatically collecting clinical data from one or more medical devices in a healthcare facility is described, comprising at least one identification device of the patient, control means of the management system, and storage means of the clinical data in the medical record, wherein the control means comprise an interface device able to operatively connect the identification device to the medical devices, the control means are able to automatically collect the clinical data and to store them in the storage means when the identification device correctly identifies the identity of the patient and the identity finds the match of the patient in the information systems of the healthcare facility, and when the interface device identifies the activation of one of the medical devices provided for in one of the active prescriptions of the patient.

The present invention relates to a system and method for managing the medical record of a patient by automatically collecting clinical data from one or more medical devices in a healthcare facility.

Healthcare facilities have currently reached a good level of computerization and a large part of the clinical data of the patients, whether they are hospitalized (“in-patients”) or external (“out-patients”), is stored in digital format in the archives of the facilities themselves.

However, healthcare systems suffer from a so-called “scattered” digitization, which does not cover all medical specialties and gives way to possible data duplication and errors due to the manual re-entering of the data. Indeed, at present, a series of mutually independent data “islands” is observed, in which the information is modeled to meet specific needs, almost exclusively in the restricted environment of the clinical staff and internal processes of the involved organizations, which is a direct result of the high compartmentation of such processes and organizations. Such a structuring in islands does not allow to managing patients optimally, since data available in one of them may also be useful to another one which lacks them, and therefore a manual transmission of data must be used, with possible resulting errors due to the mismatch between patients, for example.

Thereby, the traceability and safety of all clinical processes is not ensured and the possibility of a computerized access to the entire medical history of a patient is limited.

Furthermore, even if data flow in digital format within the different islands, it is difficult to ensure the exactness, correctness and consistency of data: data collection and the performing of medical procedures are solely entrusted to the good will and experience of the operators. This does not allow to ensure that the cycle of prescribing, identifying the patient and administering-performing medical procedures is correctly carried out at each step. It is for this reason that key operations, such as the positive, i.e. correct, identification of the patient and of the operator, the time when the medical procedure must be performed, the applied procedures, and so on, require systems which avoid human errors by means of automatic and traceable mechanisms. Specifically, the correct identification of the patient within the information systems is now more than ever a crucial aspect, not only for the safety issues arising therefrom and the need for interoperability, but also for the exactness of the data collected: data achieve a true potential only if they are accurate and associated with the correct patient. A study by CHIME (College of Healthcare Information Management Executives) has shown how errors due to an incorrect identification of the patient range from 8 to 20%, with 19% of the surveyed hospitals admitting to having had at least one adverse event during the previous year due to patient identification issues.

Another development direction, which has recently been taken, is the paradigm of “Patient Empowerment”, the key principle of which is to make patients more responsible for their health, shifting to a model in which healthcare professionals are now collaborating with the patient in defining the treatment paths rather than taking unilateral decisions on his/her behalf as it occurred in the past. With an approach of this type it is therefore essential that the patient has the full availability of his/her clinical information, on time and by means of the most congenial communication channels, and becomes the true owner of the data relating to his/her own health. The patient must indeed be able to make conscious decisions by accessing all the necessary information, structured in an understandable and usable format. At present, access to these data is not always ensured and is often difficult: data are in fact stored in a piecemeal fashion in the different healthcare facilities, sometimes digitally and sometimes in hardcopy and in a multitude of different formats, thus making impossible for the patient to have one single container where to find his/her own medical history easily.

Lastly, a strong mobility of patients is currently observed, across different healthcare facilities, both within their own country of residence and outside. Because of the criticalities previously described, the patient has no possibility to transfer data across the different facilities, both because there are no archives containing all the medical history of the patients and because the data are not structured so as to allow to export and convert them into languages and formats other than the source ones.

It is the object of the present invention to provide a system and method for managing the medical record of a patient by automatically collecting clinical data from one or more medical devices in a healthcare facility. In particular, it is the object of the present invention to obtain an automated and traceable process for the collection, storage and use of clinical data, which starts from the correct identification of the patient. The main premise of such a process is the right of the patient to have his/her own medical data available; this is made possible by integrating existing tools in healthcare facilities with the devices manufactured by the Applicant for the traceability of clinical processes.

All clinical data must be collectable and transferable without the need for manual intervention by the operators; there must then be the possibility, upon authorization by the patient, to save the clinical data so that they are globally accessible by the patients, by means of different types of devices and applications. Patients may then give consent to the use of all data, or a part thereof, to specific recipients (for the purposes, for example, of sharing life-saving information in emergency situations, or to avoid the repetition of tests already performed in other facilities, etc.).

This and other objects are achieved by a management system according to claim 1. In particular, the aforesaid objects are achieved by a system for managing the medical record of a patient by automatically collecting clinical data from one or more medical devices in a healthcare facility, the healthcare facility being provided with information systems able to store at least the personal data and the active prescriptions of each patient, the management system is characterized in that it comprises at least one identification device of the patient, control means of the management system and storage means of the clinical data in the medical record, where the control means comprise an interface device able to operatively connect the identification device to the medical devices, the control means are able to automatically collect the clinical data and to store them in the storage means when the identification device correctly identifies the patient and the identity finds the match of the patient in the information systems of the healthcare facility, and when the interface device identifies the activation of one of the medical devices provided for in one of the active prescriptions of the patient.

In a further aspect, the aforesaid objects are achieved by a method for managing the medical record of a patient by automatically collecting clinical data from one or more medical devices in a healthcare facility, the healthcare facility being provided with information systems able to store at least the personal data and the active prescriptions of each patient, the management method is characterized in that it comprises the following steps:
identifying, by means of an identification device, the identity of the patient;
matching, by control means, the identified identity of the patient with the master data present in the information systems;
retrieving, by the control means, the active prescriptions in the information systems when said patient is matched;
identifying, by the control means, the activation of a medical device in the healthcare facility, the medical device being operatively connected to the identification device by means of an interface device;
transferring, by the control means, the clinical data from the medical device, and storing, by the control means, the clinical data in the medical record in storage means, if the identified medical device belongs to one of the active prescriptions of the patient.

These and other features of the present invention will become more apparent from the following detailed description of embodiments thereof, shown for illustrative and non-limiting purposes, in the accompanying drawings, in which:

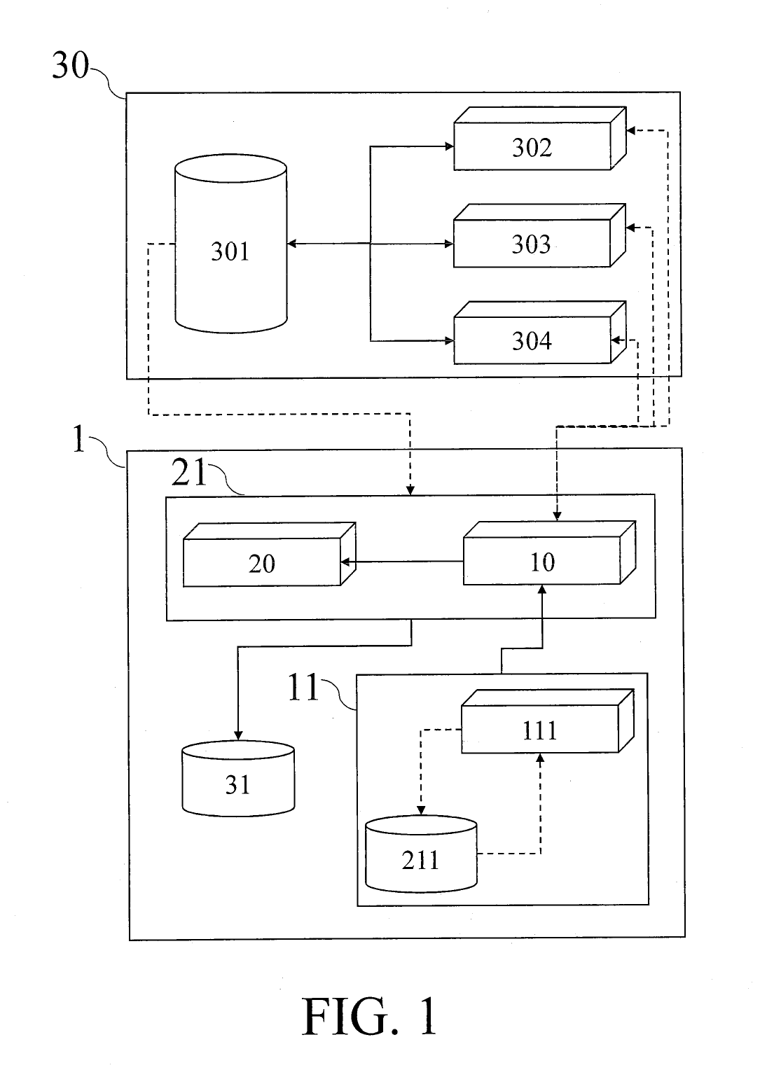

FIG. 1 shows a preferred embodiment of the system for managing the medical record of a patient according to the present invention;

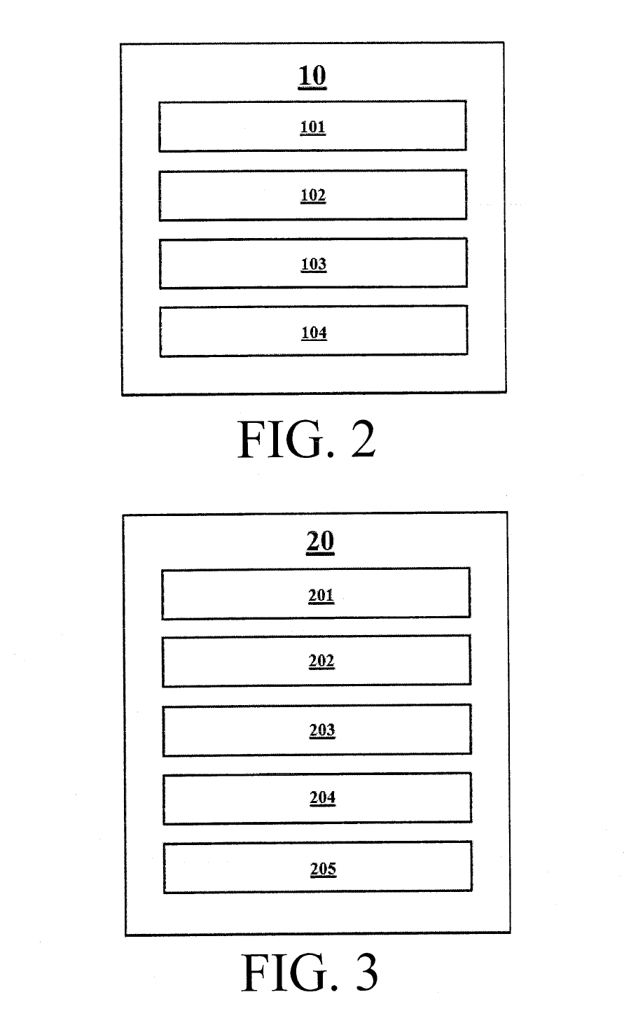

FIG. 2 shows a block diagram of an interface device of the embodiment in FIG. 1;

FIG. 3 shows a block diagram of a device for managing the clinical data flow of the embodiment in FIG. 1;

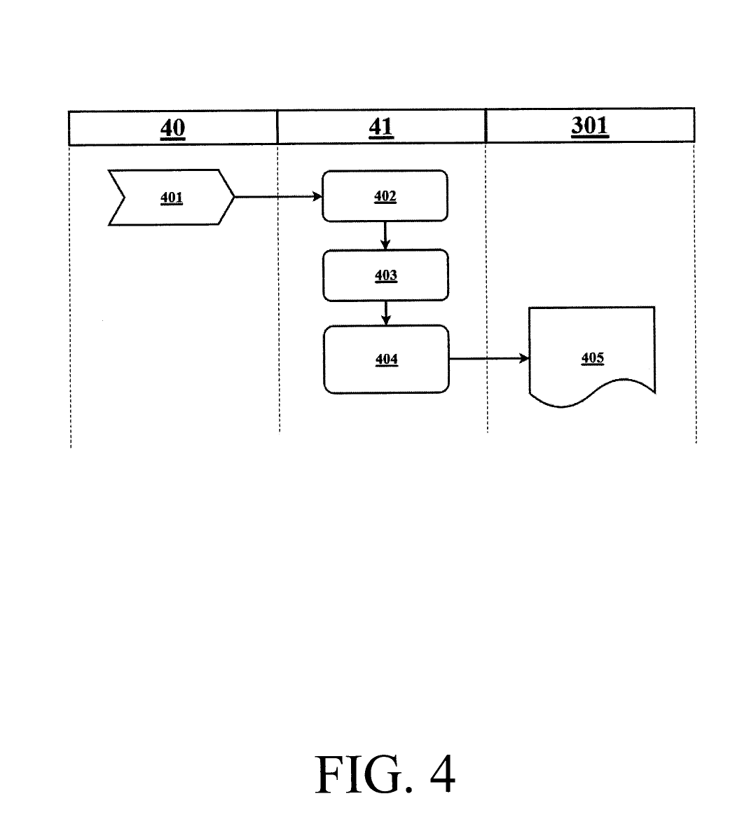

FIG. 4 shows a block diagram of a known process flow relating to the input of a prescription in the healthcare information systems;

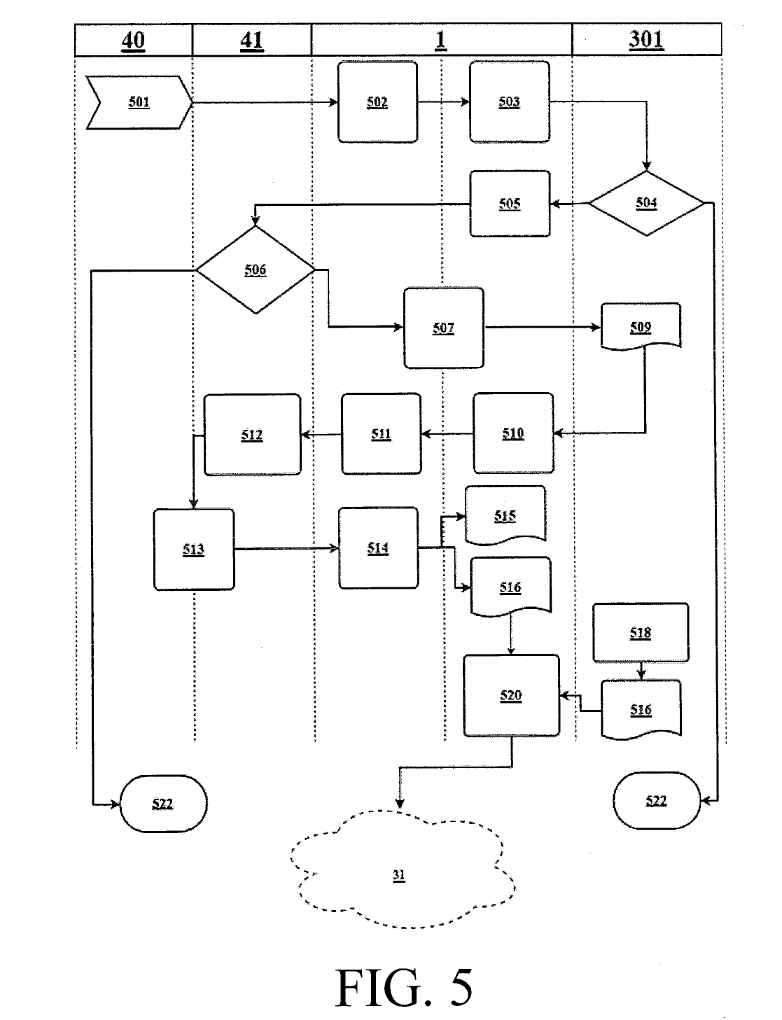

FIG. 5 shows a block diagram of the embodiment in FIG. 1;

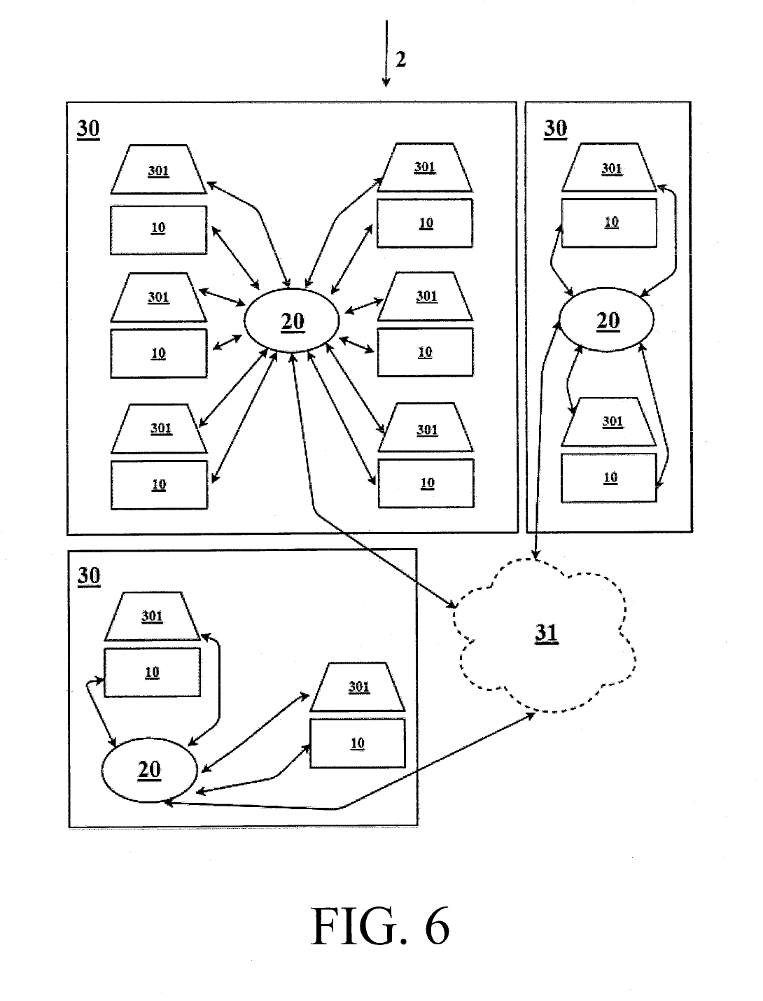

FIG. 6 shows a second embodiment of the system for managing the medical record of a patient according to the present invention.

In the exemplary depiction in FIG. 1, the preferred embodiment of system 1 for managing the medical record of a patient is shown. Such a medical record is obtained by automatically collecting clinical data from medical devices 302, 303, 304 arranged within a healthcare facility 30, shown by way of example with a number of the elements useful to the description of the present invention.

The term healthcare facility means, in the present invention, a set of welfare functions, activities and services provided by public or private facilities. By way of non-limiting example, healthcare facilities are hospitals, laboratories, sample collection centers.

The healthcare facility 30, shown in FIG. 1, further comprises at least one information system 301 operatively connected to the medical devices 302, 303, 304 of the healthcare facility 30. Such an information system 301 may comprise the Laboratory Automation System (LIS), the Anatomical Pathology Laboratory Automation System (APLIS), the Emergency Information System (EIS), the Pharmacy Information System (PIS) and the Hospital Information System (HIS). Alternatively, the aforesaid information system may consist of one of the aforesaid systems or of a portion thereof. In any case, the information system 301 (or the information systems provided, if separate) stores a plurality of information relating to the activities performed on the patients of the healthcare facility 30, including their personal data and the active prescriptions for each patient. The connection between the information system 301 and the medical devices 302, 303, 304 allows, for example, to store the clinical data from the aforesaid medical devices 302, 303, 304 in the information system 301 of the healthcare facility 30.

According to the present embodiment, the medical devices are an apparatus for traceably labeling tubes 302, a vital signs monitor 303, and a diagnostic imaging device 304, respectively. In further embodiments (not shown), the medical devices may comprise only one or more of the aforesaid devices or further medical devices provided with an electrical/electronic connection.

The term active prescription means a medical prescription which has not yet been performed on the patient for whom the aforesaid prescription is intended. In particular, the prescription is a healthcare program carried out, i.e. prescribed, by an authorized person, such as a doctor or a healthcare operator, in the form of instructions which govern the treatment plan of the individual patient. Once the treatment plan has been carried out, the prescription is no longer active. The prescription may include using diagnostic tools or active administration tools, more generally medical devices, in particular electronic medical devices.

System 1 for managing the medical record of a patient, according to the present invention, provides for the automatic collection of clinical data from one or more medical devices 302, 303, 304 arranged within the aforesaid healthcare facility 30.

In particular, the management system 1 according to the present invention comprises at least three main elements: a patient identification device 11, control means 21 for controlling the entire management system 1, and storage means 31 for storing the clinical data in appropriate medical records, one for each patient. Therefore, such a system 1 for managing the medical record of a patient is associated with the healthcare facility 30, in particular with the information system 301 and the medical devices 302, 303, 304 connected thereto.

In further detail, the control means 21 comprise an interface device 10, able to operatively connect the identification device 11 to the medical devices 302, 303, 304. Additionally and preferably, the aforesaid control means 21 comprise a management device 20 for managing clinical data which is operatively connected to the aforesaid interface device 10.

Therefore, within the healthcare facility 30, at least one interface device 10 is included, being associated with the information system 301 and with one or more of the medical devices 302, 303, 304 provided within the aforesaid healthcare facility 30. In the embodiment shown in FIG. 1, the interface device 10 connects all the medical devices 302, 303, 304 present within the facility. Such a connection is achieved by means of a bi-directional connection between the parties. Similarly, the connection may be of the mono-directional type. The interface device 10 comprises a UI (user interface) 101 which allows to manage 102 the medical devices 302, 303, 304, to manage 103 the identification devices 11 associated therewith, and to communicate 104 the clinical data and log data (FIG. 2). The activities carried out by the interface device 10 may thus be carried out by means of a single UI 101 or by means of a plurality of UIs, each assigned to one or more of the aforesaid management or communication activities.

In a further embodiment (not shown), the interface device, in addition to the function of connecting and interfacing the medical devices to the management system, is further able to activate the aforesaid medical devices. In particular, such an activation is achieved when the identification device correctly identifies the identity of the patient, and such an identity finds the match of the patient in the information systems of the healthcare facility, and when the control means identify the medical device to be activated within one of the active prescriptions.

In the preferred embodiment, shown in FIG. 1, the control means 21 further comprise a management device 20 of clinical data. Such a management device 20 of clinical data provides for the management 201 of the interface device 10, for specific domain operations 202, for the integration 203, the standardization 204, and the communication 205 of the clinical data (FIG. 3). The connection and functions of the management device 20 will be detailed below.

The connection between the management system 1 and the information system 301 of the healthcare facility 30 is achieved by means of a further bi-directional connection with the control means 21. Such a connection may even be of the mono-directional type only. In a further embodiment (not shown), the connection between the management system 1 and the aforesaid information system 301 may be directly achieved by at least one of the components of the control means 21, preferably by the interface device 10.

Therefore, the control means 21 are able to automatically collect and to store, in the storage means 31 of the management system 1, the clinical data from one or more of the aforesaid medical devices 302, 303, 304. In particular, the automatic collection and the corresponding storage of the clinical data are achieved upon the correct identification of the identity of the patient, by means of the identification device 11, when the aforesaid identity finds the match of the patient within the information systems 301 of the healthcare facility 30. Furthermore, the automatic collection and storage operations are carried out when the interface device 10 identifies the activation of one of the medical devices 302, 303, 304 provided for in one of the active prescriptions of the patient.

The collection of the clinical data must always start from the correct identification of the patient, which comprises the correct identification of the identity of the patient and the correct matching of the aforesaid identity within the master database embedded in the information systems 301 of the healthcare facility 30. For illustrative but non-limiting purposes, the identification device 11 may use one or more biometric identification technologies, such as fingerprint, iris, retina, vascular pattern, palm print, and face recognition. In the preferred embodiment, shown in FIG. 1, the identification device 11 comprises a biometric recognition device 111 for the patient 40 and at least one identification information system 211 for storing and identifying the identities of the patients undergoing biometric recognition. The identification device 11 is thus connected to the medical devices 302, 303, 304 of the healthcare facility 30 by interposing the control means 21, and in particular the interface device 10. This allows the plural connection of the medical devices 302, 303, 304 to a single identification device 11 when the aforesaid medical devices 302, 303, 304 are arranged close to one another. The components of the identification device 11 are thus operatively connected one another by means of a connection of the bi-directional type, although the physical arrangement of the elements may be different. In detail, the information system 211 is distinct and separate from the information system 301 of the healthcare facility 30.

In a further embodiment (not shown), the management system may comprise a plurality of identification devices each operatively connected to a single medical device with the interposition of a corresponding interface device. For example, this may be required due to the arrangement of the medical devices in different departments or physical locations of the same healthcare facility. In this case, the system for managing the medical record of a patient will need to provide for an interface device duplication, so as to ensure the coupling to each identification device and the corresponding medical device(s) associated therewith. In this case, the identification information system is preferably implemented so as to be unique and connected to all biometric recognition devices, although arranged in a position not physically close to the aforesaid devices.

FIG. 4 describes a known process for inputting a prescription into the healthcare information systems, which simply implies the patient 40 to request 401 a visit, the doctor 41 to accept 402 the patient 40, to perform 403 the visit and the prescription 404 (for example drugs, specialist visit, tests). The process ends with the recording 405 of the prescription in the information system 301 of the healthcare facility 30 or of an upper level information system which can be accessed by such a healthcare facility 30. The recorded prescription becomes an active prescription until the expected activities are not fully completed.

A method for managing the medical record of a patient (FIG. 5) by automatically collecting clinical data from one or more medical devices 302, 303, 304 arranged within the aforesaid healthcare facility 30 is described below. Such a method is described in successive steps, by employing the system 1 for managing the medical record in its first embodiment.

As shown in FIG. 5, the arrival of the patient 40 at the healthcare facility 30 triggers a step 502, 503 of identifying him/her. This is performed, in a first step 502, by the identification device 11 included within the system 1 for managing the medical record of the patient 40 him/herself. In this regard, the identification device 11, and in particular the biometric recognition device 111, identifies at least one biometric item of the patient 40, for example the fingerprint or the iris, and generates a unique identifier of the patient 40. The recognition is carried out by means of a query, which converts either the fingerprint or the iris into an ID “Repository”. In an alternative embodiment, the ID may already be contained in an object identifying the patient, for example an identification smart card. The identifier of the patient 40 is transmitted to the identification information system 211 to perform the identification request 503 of the patient's identity.

In a second step, the matching 504 of the identified identity of the patient 40 with the master data present within the information systems 301 is carried out by the control means 21. In case of confirmation of the identity, the latter is sent to the information systems 301 of the healthcare facility 30 to obtain a confirmation 504 of the matching of the identity of the patient 40 within the healthcare facility 30 itself. If successful, the complete identification data of the patient 40 (e.g. name, surname, social security number) are forwarded 505 the control means 21 of the management system 1.

In a further embodiment, such a step may be carried out directly within the healthcare facility. For example, the patient identifier may be sent to the control means for requesting the information system of the healthcare facility to provide for an identification.

If there is no match in the data of the patient 40, the process stops 522, as the patient 40 is not matching within the information systems 301 of the healthcare facility 30, therefore the patient is not correctly identified. This situation must be considered as being totally remote, and is treated according to traditional procedures. If instead the identity matching is verified, and therefore the data of the patient 40 are returned to the control means 21, these may then be sent to the doctor 41 within the healthcare facility 30, who may further verify 506 the identity of the patient 40. Upon completion of the correct identification activities, the data are displayed on the screen of the management system 1.

In a third step, the recovery 507 of the active prescriptions 509 within the information systems 301 is performed by the control means 21, when said patient is matched, i.e. correctly identified and corresponding within the aforesaid information systems 301. The control means 21 identify, from the aforesaid active prescriptions 509, the list of the medical devices 302, 303, 304 with which the patient 40 shall interact, forwarding 510 the aforesaid list to the management system 1 so as to allow the continuation of the next steps. Such a list may be further displayed 511 on the screen of the management system 1 so as to allow the displaying 512 for the doctor 41 present within the healthcare facility 30.

In general, these may be medical devices which, when driven, do not require any manual operation by a user/medical staff; alternatively, the user may be required to give instructions. In the last case in point, the control means may send the instructions to the medical staff, so as to teach them, step by step, the correct definition of the operational flow.

Once the performance 513 of the medical procedure prescribed (automated) has been completed, the management system 1 is able to collect, i.e. to receive or request, the clinical data relating to the medical device 302, 303, 304 employed (i.e. the results of the medical procedure, such as a pressure, temperature, or blood oxygen measurement). At this point, two different scenarios arise: the result of the medical procedure may be immediate, or the result of the medical procedure is obtained after a more or less long period of time (e.g. the result of the medical procedure relating to the collection of a blood sample).

The architecture of the system 1 for managing the medical record of a patient 40 shown in FIG. 1 allows to operate in both scenarios sharing the same sequence of steps. At first, a step (not shown) of identifying the activation of a medical device 302, 303, 304 arranged within the healthcare facility 30 and connected to the management system 1 is carried out by the control means 21. A transfer step 514, again by said control means 21, then transfers the clinical data 516 from the medical device used to the management system 1 and proceeds with the storage step 517 within the storage means 31 of the system. The clinical data 516 are thus stored within the medical record of the patient 40 when the medical device identified belongs to one of the active prescriptions of the aforesaid patient 40. Therefore, in the case of the first scenario, when the result of the medical procedure is immediate and the patient 40 is near the identification device 11 or the interface device 10, the clinical data relating to the result of the medical procedure is immediately transferred to the management system 1 and, by the control means 21 (e.g. by means of the interface device 10 or by means of the device 20 for managing clinical data flow), sent to and stored within the storage means 31 of the management system 1. In addition, the aforesaid clinical data may also be sent to the information system 301 of the healthcare facility 30 for storage purposes or further operations.

It is worth noting that it is not the clinical staff that transfers the clinical data 516 and/or the log data relating to the medical procedure, but such data directly arrive to the interface device 10 which drives the aforesaid medical devices. By way of example, the drivable medical devices are for example the apparatus described in Italian patent No. 1409955 by the Applicant, an apparatus for traceably labeling tubes, a vital signs monitor, a diagnostic imaging device. The latter may include the driving of CT scanners, MRI devices or medical radiology devices.

In the case of the second type of scenario, the interface device 10 first reports the activation of one of the medical devices 302, 303, 304 employed. The control means 21, or the interface device 10 itself, then proceed with the recording of the activation of the aforesaid medical device on the medical record of the patient 40 within the storage means 31. Finally, the device 20 for managing clinical data at a later step queries the information systems 301 of the healthcare facility 30 to retrieve the clinical data of the results obtained. Such a step is depicted by way of example by step 518 of the block diagram in FIG. 5. Thereby, the management system 1 is able to retrieve at a later time the clinical data relating to the result, by suitably re-associating it with the patient 40 correctly identified, even if said patient is no longer present.

In both the first and second scenarios, once the clinical data 516 has reached the management system 1 (either immediately, or subsequently rejoined, if before the results of the medical procedures are to be awaited for), it provides for the operation 520 of standardizing the aforesaid clinical data 516, so as to uniform for example the detections of the same parameter, which detections may be carried out with different measurement units.

The standardized data 520 are then transferred from the control means 21, for example by means of interface device 10 or management device 20, to the storage means 31. Therefore, it should be noted that the control means 21 always deal with transferring data, not with storing them. The clinical data never remains, for any reason, stored in the control means 21. This is consistent with the fact that the control means 21 may be arranged within the perimeter of the healthcare facility 30, while the clinical data 516 of the patient 40 must be stored in a repository, such as the storage means 31, where the patient 40 himself/herself has authorized the storing thereof (for his/her future consultation or that of other people).

In a further embodiment, the control means, and in particular the interface device, may drive medical devices of various kind capable of performing automated medical procedures (e.g. a pressure meter, an oxygen saturation meter, etc.). According to this further embodiment, the control means, and in particular the interface device, may directly perform the activation of the medical devices after the identification, matching and recovery steps previously described. Such a step of activating the medical device is carried out when the identification device correctly identifies the identity of the patient (identification step) and said identity finds the match of the patient within the information systems of the healthcare facility (matching step), and when the control means identify the medical device to be activated in one of the active prescriptions (recovery step).

The control means 21, and in particular the interface device 10, may further initiate the generation of log data 515 which allow the traceability of the medical procedure in both scenarios (i.e. who carried out what operation, on which patient, at what time, according to which procedure and with which medical devices). Such a generation of log data 515 is carried out when the identification device 11 correctly identifies the identity of the patient 40 and finds the match of said identity within the information systems 301 of the healthcare facility 30.

In the embodiments previously described, the control means 21 thus preferably comprise an interface device 10 and a management device 20 for managing the clinical data flow. Such an interface device 10 preferably has the task to collect automatically the clinical and/or log data from the medical devices of the healthcare facility 30 or from the investigations by the medical staff. Such clinical data may be sent to the aforesaid flow management device 20 or may be managed directly by the interface device 10. For illustrative but non-limiting purposes, the interface device 10 may use connection technologies such as Wi-Fi, Bluetooth, Ethernet, Serial Ports, etc., and data protocols such as HL7, ASTM, DICOM, LOINC, ICD, etc.

Preferably, the management device 20 of the clinical data flow is the manager of the clinical data and/or log data from different sources (e.g. different medical devices or different information systems), and provides for their collection, standardization and sending to storage means. The standardization operation consists in separating the structure of the data from the data itself, so as to manage the data in healthcare systems distributed around the world in a universal manner, as well as to manage the evolution of the data over time effectively. For example, through the standardization, the data from different healthcare facilities, therefore expressed in different formats and languages, may be compared.

The management device 20 of the clinical data flow transfers the clinical data and/or log data, once standardized, to the storage means 31 for use by the patients 40, who may display, transfer, integrate or delete their own records. By way of example, the storage means 31 may comprise different types of Cloud, servers, etc.

The patient 40 may allow other players to access all his/her own clinical and/or log data or a part of them. For example, family members, staff of the emergency-urgency departments, the family doctor, etc. may be authorized.

Access to the data of the storage means 31 may take place for example by means of specific applications or web systems specifically developed, and by means of different devices (PCs, tablets, smartphones, etc.). Typically, one connects to the storage means 31 by typing a specific IP address and, logging in with appropriate credentials previously obtained, has access to the data. In this regard, the access to such data may be distinguished in two distinct although complementary manners. Indeed, a first method for the controlled sharing of data by each patient 40 may be configured, so that he/she can decide to which other players, in addition to himself/herself, give access to his/her own clinical data. For example, the patient 40 may decide to give access to his/her medical records, kept in the storage means 31, to his/her own family doctor, to parents or relatives, to accredited emergency care institutions which need to provide assistance to the patient in a state of unconsciousness (as described below), but also to some pharmaceutical companies, so as to enter in one of the so-called “Precise Medicine” programs aiming at bringing together people suffering from the same disease, so as to offer them consistent care, based on past experiences of other patients 40. The second method concerns instead the possibility to access the data of a patient 40 contained in the storage means 31 in particular emergency situations, always by other players only if explicitly authorized by the patient 40. An example is the situation where a patient 40, in a foreign country, suffers an accident which causes a state of unconsciousness: by accessing his/her medical record in the storage means 31, the emergency room operators who take care of him/her may access the so-called “Emergency Patient Summary” (EPS), which contains various historical information on the patient 40 and is intended to help the aforesaid operators to treat him/her in the most appropriate manner. Such information may comprise for example any allergies from which the patient 40 suffers, or implants present in his/her body, chronic diseases, particular drugs he/she is taking or major surgery undergone during his/her life.

A second embodiment of the system 2 for managing the medical record of a patient according to the present invention is shown in FIG. 6.

The embodiment is shown with reference to a plurality of healthcare facilities 30 (hospitals, laboratories, sample collection centers). Each healthcare facility 30 internally comprises a plurality of interface devices 10, each able to interface an identification device 11, preferably of the biometric type, in order to allow the correct identification of the patient, as previously described. Each interface device 10 is further able to interface one or more medical devices present in each of the healthcare facilities 30. The embodiment in FIG. 6 further includes a single management device 20 of clinical data flows operatively connected to all the interface devices 10 present in each healthcare facility 30. Therefore, each healthcare facility 30 is provided with a plurality of interface devices 10, and with the corresponding identification devices 11, but with a single management device 20 of the data flows. Preferably, the number of identification devices 11 is equal to the number of “Points of Care” within the healthcare facility 30, or the number of points of contact of the patient with a medical device. It should be noted that the storage means 31 provided for such an embodiment comprise a single storage means for all the healthcare facilities 30 connected to one another by the management system 2. Thereby, the medical record of the patient 40 may be fed with data from a medical device connected to the management system 2 regardless of the healthcare facility 30 providing the service.

The description of the management system 2 shown in FIG. 6 will not be further detailed, as the operation resumes, mutatis mutandis, the previous disclosure given for the described embodiments, and in particular the previous disclosure of the management system 1 and the related method in the preferred embodiment.

The management device 20 may be further used for interfacing all interface devices 10 with the information systems 301 of the healthcare facility 30, thus allowing the automatic collection, standardization and sending of the data (collected by the interface devices 10 with reference to each individual patient 40). Data are sent to appropriate storage means 31, previously described for the preferred embodiment, to be subsequently used by the patient 40 him/herself, or by other players.

Therefore, the present invention allows personal clinical data to be accessed personally, while allowing at the same time to transfer them by providing other individuals with the appropriate authorizations for accessing them.

Therefore, the innovative aspect of the present invention is that of having a system and method for managing the medical record of a patient by automatically collecting clinical data from one or more medical devices in a fully secure, automated and traceable manner. Thereby, the collected data are made available to patients or others, by collecting them in appropriate, globally accessible storage means, in particular of the Cloud type.

As regards the input of data, the innovative aspect is that it is never carried out manually by the operator, since all processes are carried out in a fully automated manner as mentioned above. The resulting traceability of the clinical data also allows to eliminate almost completely the possibility of error with respect to the detection thereof, thus overcoming the problem of lack of exactness of the data, typical of the known solutions.

Moreover, these are “certified data”, i.e. generated by a doctor in the context of the contact occurring between a patient and one or more healthcare facilities which detect his/her data; it is of particular importance in this sense the fact that, in a single solution, both the data originating from a prolonged contact of the patient with a healthcare facility (such as a hospitalization) and those originating instead from an extemporaneous contact (e.g. the collection of biological material which is carried out in a few minutes) may be integrated, thus covering both “in-patient” and “out-patient” operations.

Furthermore, data are appropriately standardized, so as to make them usable in a consistent manner to anyone who needs to interpret them, in every part of the world.

The accessibility to personal data is also ensured to patients anytime, anywhere, by means of dedicated applications or the web. Moreover, the same data may be shared, in whole or in part, with healthcare operators or trusted persons, in view of the immediate availability thereof for the latter in emergency situations.

The management system according to the present invention may be used by a legal entity to provide a costly service rendered to citizens or to an institution which, on behalf of the citizen him/herself, takes on, authorized by the same, the provision of said service.

1. A system for managing the medical record of a patient by automatically collecting clinical data from one or more medical devices in a healthcare facility, said healthcare facility being provided with information systems able to store at least the personal data and the active prescriptions of each patient, said management system it comprises at least one identification device of said patient, control means of said management system, and storage means of said clinical data in said medical record, wherein said control means comprise an interface device able to operatively connect said identification device to said medical devices, said control means are able to automatically collect said clinical data and to store them in said storage means when said identification device correctly identifies the identity of said patient and said identity finds the match of said patient in said information systems of said healthcare facility, and when said interface device identifies the activation of one of said medical devices provided for one of said active prescriptions of said patient.

2. The system for managing the medical record of a patient according to claim 1, wherein said interface device is able to activate said medical devices when said identification device correctly identifies said identity of said patient and said identity finds the match of said patient in said information systems of said healthcare facility, and when said control means identify said medical device to be activated in one of said active prescriptions.

3. The system for managing the medical record of a patient according to claim 1, wherein said control means record, on said storage means, the activation of one of said medical devices in said medical record of said patient.

4. The system for managing the medical record of a patient according to claim 3, wherein said control means comprise a management device of said clinical data, said management device is operatively connected to said interface device and to said information systems of said healthcare facility, said management device of said clinical data is able to query said information systems of said healthcare facility when the activation of one of said medical devices is recorded in said medical record of said patient in order to automatically collect said clinical data from said information systems of said healthcare facility.

5. The system for managing the medical record of a patient according to claim 1, wherein a plurality of said interface devices, each able to interface an identification device with one or more medical devices, and a management device of said clinical data operatively connected to all of said interface devices.

6. The system for managing the medical record of a patient according to claim 1, wherein said storage means are able to be queried in order to use said clinical data of said medical record of said patient at any time and from anywhere in the world.

7. The system for managing the medical record of a patient according to claim 1, wherein said identification device comprises at least one biometric recognition device of said patient and at least one identification information system for storing and identifying said identities of said patients, said identification information system being distinct and separated from said information system of said healthcare facility.

8. The system for managing the medical record of a patient according to claim 1, wherein said control means are able to initiate the generation of log data and the recording of said log data in said storage means, when said identification device correctly identifies the identity of said patient and said identity finds the match of said patient in said information systems of said healthcare facility.

9. The system for managing the medical record of a patient according to claim 1, wherein said medical devices of said healthcare facility comprise an apparatus for traceably labeling tubes and/or a vital signs monitor and/or a diagnostic imaging device.

10. A method for managing the medical record of a patient by automatically collecting clinical data from one or more medical devices in a healthcare facility, said healthcare facility being provided with information systems able to store at least the personal data and the active prescriptions of each patient, said management system is characterized in that it comprises the following steps:
identifying, by means of an identification device, the identity of said patient;
matching, by control means, the identified identity of the patient with the master data present in said information systems;
retrieving, by said control means, the active prescriptions in said information systems when said patient is matched;
identifying, by said control means, the activation of a medical device in said healthcare facility, said medical device being operatively connected to said identification device by means of an interface device;
transferring, by said control means, the clinical data from said medical device, and storing, by said control means, said clinical data in said medical record in storage means, if said medical device identified is provided for in one of said active prescriptions of said patient.

11. The method for managing the medical record of a patient according to claim 10, wherein the step of identifying the identity of said patient comprises detecting at least one biometric feature of said patient, and generating a unique identifier of said patient.

12. The method for managing the medical record of a patient according to claim 10, further comprising the step of activating, by means of said interface device, one of said medical devices in said healthcare facility, when said control means control the activation of a medical device provided for in one of said active prescriptions of said patient after said step of retrieving said active prescriptions.

13. The method for managing the medical record of a patient according to claim 10, wherein said method is implemented by means of a system for managing the medical record of a patient according to a system by automatically collecting a system for managing the medical record of a patient by automatically collecting clinical data from one or more medical devices in a healthcare facility, said healthcare facility being provided with information systems able to store at least the personal data and the active prescriptions of each patient, said management system it comprises at least one identification device of said patient, control means of said management system, and storage means of said clinical data in said medical record, wherein said control means comprise an interface device able to operatively connect said identification device to said medical devices, said control means are able to automatically collect said clinical data and to store them in said storage means when said identification device correctly identifies the identity of said patient and said identity finds the match of said patient in said information systems of said healthcare facility, and when said interface device identifies the activation of one of said medical devices provided for one of said active prescriptions of said patient.
